(19) United States
(12) Patent Application Publication
(10) Pub. No.: US 2026/0341424 A1
(43) Pub. Date: Oct. 29, 2026

(54) BIOMARKERS, SYSTEMS, AND METHODS FOR DETECTING RESECTABLE PANCREATIC DUCT ADENOCARCINOMA

(71) Applicant: PRECOGIFY PHARMACEUTICAL CHINA CO., LTD., Beijing (CN)

(72) Inventors: Kai LIN, Beijing (CN); Xudong DAI, Beijing (CN); Yu TIAN, Beijing (CN); Zhiqiang MA, Beijing (CN); Tengsong CUI, Beijing (CN)

(73) Assignee: PRECOGIFY PHARMACEUTICAL CHINA CO., LTD., Beijing (CN)

(21) Appl. No.: 19/687,049

(22) Filed: May 25, 2026

Related U.S. Application Data
(63) Continuation-in-part of application No. PCT/CN2023/134494, filed on Nov. 27, 2023.

(51) Int. Cl. G16C 20/70; G16H 50/20

(52) U.S. Cl. CPC G16C 20/70; G16H 50/20

(57) ABSTRACT

The present disclosure provides a panel of serum metabolites biomarkers usable for diagnosis of resectable pancreatic duct adenocarcinoma (PDAC). A method for detecting resectable PDAC using the serum metabolites biomarkers is also provided. For example, the method provided by the present disclosure is a non-invasive approach that may utilize serum samples for detecting resectable PDAC. Moreover, the method for detecting resectable PDAC may detect PADC of different stages (e.g., stage I, stage II). A kit for detecting resectable PDAC is also provided.

### CROSS-REFERENCE TO RELATED APPLICATIONS

This application is a continuation-in-part of International Patent Application No. PCT/CN2023/134494, filed on Nov. 27, 2023, the contents of which are hereby incorporated by reference.

### TECHNICAL FIELD

The present disclosure generally relates to detection of pancreatic duct adenocarcinoma (PDAC), and in particular, to biomarkers, systems, and methods for detecting resectable PDAC.

### BACKGROUND

Pancreatic duct adenocarcinoma (PDAC) and pancreatic duct cancer generally refer to the same type of cancer, which originates in the cells lining the pancreatic ducts. Resectable PDAC refers to a stage of this cancer that is amenable to surgical resection, and it implies that the tumor is confined to the pancreas and has not spread to nearby blood vessels or distant organs, making it potentially treatable or even curable through surgical intervention. PDAC that is considered not resectable, on the other hand, has a very poor prognosis with a 5-year survival rate of only 6%. This is largely due to the late detection of pancreatic cancer with 80%-85% of patients being diagnosed in unresectable stages, while 5-year survival rate could increase to 42% if the patients could be diagnosed at localized stages (resectable stages). Thus, there is a clear medical need for the early detection of resectable PDAC patients.

Commonly used diagnostic methods of PDAC include transabdominal ultrasound, various blood tests and trans-sectional imaging. The best-established marker for blood test for PDAC detection is carbohydrate antigen 19-9 (CA19-9). However, this biomarker is also elevated in pancreatitis, a disease which improves the risk of having this cancer, thus further hampering the accuracy for early detection of PDAC (current diagnostic accuracy between 50% and 60%). Therefore, it is desirable to find new biomarkers that can efficiently discriminate between resectable PDAC and pancreatitis patients, thereby significantly reducing PDAC induced mortality.

### SUMMARY

According to an aspect of the present disclosure, a system for detecting resectable pancreatic duct adenocarcinoma (PDAC) in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table A; (b) determining a sample score by processing the quantified abundance of each of the one or more target metabolites using a prediction model; and (c) estimating whether the subject has resectable PDAC by comparing the sample score to a cut-off score.

According to another aspect of the present disclosure, a system for detecting stage I of pancreatic duct adenocarcinoma (PDAC) in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table A; (b) determining a sample score by processing the quantified abundance of each of the one or more target metabolites using a prediction model; and (c) estimating whether the subject has stage I of PDAC by comparing the sample score to a cut-off score.

According to yet another aspect of the present disclosure, a system for detecting stage II of pancreatic duct adenocarcinoma (PDAC) in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table A; (b) determining a sample score by processing the quantified abundance of each of the one or more target metabolites using a prediction model; and (c) estimating whether the subject has stage II of PDAC by comparing the sample score to a cut-off score.

According to yet another aspect of the present disclosure, a method of detecting resectable pancreatic duct adenocarcinoma (PDAC) in a subject and treating the subject is provided. The method may include (a) obtaining, from a quantitative measurement device, quantified abundance of one or more target metabolites in a panel of a plurality of metabolites in a sample from the subject, wherein the plurality of metabolites include the metabolites of Table A; (b) determining a sample score by processing the quantified abundance of each of the one or more target metabolites using a prediction model; (c) determining whether the subject has resectable PDAC by at least comparing the sample score to a cut-off score; and (d) in response to determining that the subject has resectable PDAC, applying a treatment to the subject, wherein the treatment includes a surgery, radiation therapy, chemotherapy, targeted therapy, or immunotherapy.

According to still another aspect of the present disclosure, a use of one or more target metabolites for preparing a kit for detecting resectable pancreatic duct adenocarcinoma (PDAC) in a subject is provided. The one or more target metabolites including at least one, two, three, four, five, eight, ten, or all of the metabolites in Table A.

According to yet another aspect of the present disclosure, a use of one or more target metabolites in generating a trained machine-learning model for estimating whether the subject has resectable PDAC is provided. The one or more target metabolites include at least one, two, three, four, five, eight, ten, or all of the metabolites in Table A.

A kit for detecting resectable pancreatic duct adenocarcinoma (PDAC) in a subject is provided. The kit may include one or more reagents for quantifying one or more target metabolites in a panel of a plurality of metabolites, wherein the plurality of metabolites include the metabolites of Table A.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a schematic diagram illustrating an exemplary system for detecting PDAC in a subject according to some embodiments of the present disclosure;

FIG. 2 is a block diagram illustrating an exemplary processing device according to some embodiments of the present disclosure;

FIG. 3 shows a performance of a prediction model to discriminate resectable PDAC and non-PDAC group individuals in the training set;

FIG. 4 shows a performance of the prediction model to discriminate resectable PDAC and non-PDAC group individuals in the testing set;

FIG. 5 is a performance of the prediction model to discriminate stage I of PDAC and non-PDAC group in the testing cohort;

FIG. 6 is a performance of the prediction model to discriminate stage II of PDAC and non-PDAC group in the testing cohort;

FIG. 7A shows the ROC curve for the YX005;

FIG. 7B shows the ROC curve for the YX042;

FIG. 7C shows the ROC curve for the YX073;

FIG. 7D shows the ROC curve for the YX126;

FIG. 8A shows the ROC curve for the combination of YX005 and YX042;

FIG. 8B shows the ROC curve for the combination of YX005 and YX050;

FIG. 8C shows the ROC curve for the combination of YX005 and YX073;

FIG. 8D shows the ROC curve for the combination of YX020 and YX073;

FIG. 8E shows the ROC curve for the combination of YX020 and YX131;

FIG. 8F shows the ROC curve for the combination of YX040 and YX126;

FIG. 8G shows the ROC curve for the combination of YX042 and YX126;

FIG. 8H shows the ROC curve for the combination of YX050 and YX073;

FIG. 8I shows the ROC curve for the combination of YX050 and YX126;

FIG. 8J shows the ROC curve for the combination of YX050 and YX131;

FIG. 9A shows the ROC curve for the combination of YX005, YX006, and YX042;

FIG. 9B shows the ROC curve for the combination of YX005, YX017, and YX042;

FIG. 9C shows the ROC curve for the combination of YX005, YX039, and YX042;

FIG. 9D shows the ROC curve for the combination of YX050, YX053, and YX147;

FIG. 9E shows the ROC curve for the combination of YX050, YX054, and YX073;

FIG. 9F shows the ROC curve for the combination of YX005, YX042, and YX077;

FIG. 10 shows the ROC curve of the CA19-9 in the training cohort; and

FIG. 11 shows the ROC curve of the CA19-9 in the testing cohort.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

The present disclosure provides a group of diagnostic biomarkers usable for detecting resectable pancreatic duct cancer (PDAC) in a subject. These metabolites have important clinical value and practical significance for non-invasive diagnosis and beneficial to the precision treatment of this disease. These metabolites can be further used to monitor remission of PDAC lesions after sectioning, for example, monitor status of post-surgical patients, for example, disease alleviation, or recurrence via non-invasive blood test, instead of relying on invasive approaches. A system and method for detecting resectable PDAC using the biomarkers are also provided. For example, the system and method provided by the present disclosure utilize body fluid samples (e.g., blood serum samples) for detecting resectable PDAC in a non-invasive approach. Moreover, the system and method for detecting resectable PDAC can detect different stages of resectable PDAC (e.g., stage I and II). The detection of resectable PDAC in a subject can effectively improve the survival rate for resectable PDAC patients. As compared with conventional methods for detecting resectable PDAC (e.g., the methods using transabdominal ultrasound or various blood tests), the methods for detecting resectable PDAC provided by the present disclosure are non-invasive and are capable of effectively distinguishing subjects having PDAC from normal subjects. In addition, the biomarkers provided by the present disclosure are significantly more accurate than CA19-9 alone.

As used herein, the term “subject” of the present disclosure refers to any human or non-human animal. Exemplary non-human animals may include Mammalia (such as chimpanzees and other apes and monkey species), farm animals (such as cattle, sheep, pigs, goats, and horses), domestic mammals (such as dogs and cats), laboratory animals (such as mice, rats, and guinea pigs), or the like. In some embodiments, the subject is a human. In some embodiments, the term “normal subject” refers to a subject who is not suffering from resectable PDAC, e.g., a healthy subject, or a subject having pancreatitis. In some embodiments, the term “normal subject” refers to a subject who is not suffering from resectable PDAC and pancreatitis. Which definition of the term is used can be determined in the context.

According to an aspect of the present disclosure, a group of biomarkers usable for diagnosis of resectable pancreatic duct adenocarcinoma (PDAC) is provided.

In some embodiments, the biomarkers may be related to alternated metabolites induced by pancreatic cancer. In some embodiments, the group of biomarkers may include one or more target metabolites correlated with resectable PDAC. The one or more target metabolites may be serum metabolites that exhibit significant differentiation between a positive group of subjects (resectable PDAC) and a negative group of subjects (having normal conditions, pancreatitis). Alternatively, the one or more target metabolites may be selected from a plurality of candidate metabolites based on the performance of machine-learning models trained using one or more of the plurality of candidate metabolites. More details regarding the determination of the one or more target/candidate metabolites may be found elsewhere in the present disclosure, e.g., Examples 1 and 2.

In some embodiments, the abundance of the metabolite(s) in a sample obtained from a normal subject may be different from the abundance of the metabolite(s) in a sample obtained from a subject that has PDAC. As used herein, the term “abundance” refers to the quantity or amount of a substance in a certain sample. The sample may be a solid sample, a fluid sample, a gas sample, or the like, or any combination thereof. The solid sample may include, for example, feces, earwax, etc. The fluid sample may include the body fluid of the subject, such as blood, serum, saliva, urine, sweat, or the like, or any combination thereof. The gas sample may include flatus, breath, etc. Merely by way of example, the one or more target metabolites may be present in the serum and may be referred to as “serum metabolites”.

In some embodiments, to measure the abundance of a metabolite, the concentration or amount of the metabolite in a fluid sample (e.g., serum), a solid sample, or a gas sample (e.g., flatus) may be measured. The abundance of each of the one or more target metabolites may be quantified by a quantitative measurement device using a relative quantification approach or an absolute quantification approach. For example, the abundance of a metabolite may be a relative abundance determined based on a normalized value or a relative value with respect to a control. In some embodiments, the control may be the precise concentration or amount of a set of chemicals that are artificially added into a subject, such as spike-in control. Alternatively, the control may be the concentration or amount of the same metabolite of a sample obtained from a pool of subjects who do not have PDAC and is considered physically healthy. Alternatively, the abundance of the metabolite may be an absolute abundance that directly reflects the level of the metabolite in the subject. In some embodiments, the abundance of the metabolite may be obtained by mass spectrometry, chromatography (e.g., HPLC), and any other appropriate techniques.

Table 1 shows an exemplary group of metabolites that can be used for the diagnosis of PDAC. Each of the metabolites, which are biomarkers, has shown a strong and reliable correlation with the presence of PDAC. In some embodiments, the group of diagnostic biomarkers provided by the present disclosure may include one or more target metabolites of Table 1. In some embodiments, the group of diagnostic biomarkers may include at least one of the metabolites of Table 1. In some embodiments, the group of diagnostic biomarkers may include at least two of the metabolites of Table 1. In some embodiments, the group of diagnostic biomarkers may include at least three of the metabolites of Table 1. In some embodiments, the group of diagnostic biomarkers may include at least four of the metabolites of Table 1. In some embodiments, the group of diagnostic biomarkers may include at least five of the metabolites of Table 1. In some embodiments, the group of diagnostic biomarkers may include at least 6, 7, 8, 9, 10, 11, 12, 13, 14 or 15 of the metabolites of Table 1. As another example, the group of diagnostic biomarkers may include all of the metabolites of Table 1.

TABLE 1

| | | MASS | Delta |
|---|---|---|---|
| Meta ID | Compound | (+/−) | (ppm) |
| YX005 | C17H21N2O5 | 333.145(+) | 2 |
| YX006 | C27H46O10 | 548.338(+) | 9 |
| YX017 | C21H41NO4 | 372.321(+) | 27 |
| YX020 | C18H39NO2 | 302.305(+) | 1 |
| YX039 | C26H52NO7P | 522.362(+) | 13 |
| YX040 | C26H52NO7P | 522.356(+) | 1 |
| YX042 | C21H44NO6P | 438.3(+) | 4 |
| YX050 | C12H26NO7P | 362.113(−) | 3 |
| YX053 | C20H28O4 | 331.191(−) | 1 |
| YX054 | C22H46NO7P | 512.299(−) | 1 |
| YX073 | C25H39NO6S | 540.273(−) | 17 |
| YX077 | C29H47NO8 | 536.315(−) | 16 |
| YX126 | C20H32O3 | 319.228(−) | 0 |
| YX131 | C20H32O3 | 319.229(−) | 0 |
| YX147 | C34H64NO10P | 712.3761(−) | 28 |

Note:
MASS refers to mass-to-charge ratio; (−) represents negative charge; (+) represents positive charge.

In some embodiments, one or more of the metabolites shown in Table 1 can be used for detecting resectable PDAC in the subject. For example, mass spectrometry (or other techniques) may be used to quantify the abundance of one or more target metabolites in a panel of a plurality of metabolites in a sample. The abundance of each metabolite that has been quantified can be processed and used to detect resectable PDAC and/or facilitate the treatment of resectable PDAC in the subject. The term “treatment of resectable PDAC,” as used herein, refers to partially or totally inhibiting, delaying, or preventing the progression of cancer cells lining the pancreatic ducts; inhibiting, delaying, or preventing the recurrence of cancer including cancer metastasis; preventing the onset or development of cancer (chemoprevention) in the subject; and/or removing the PDAC. More description about treating method can be found else where in the present disclosure. In some embodiments, any one of the metabolites in Table 1 can be quantified and used for these purposes. In some embodiments, any one, two, or three metabolites in Table 1 can be quantified and used for these purposes. In some embodiments, any four, five, six, seven, eight, nine, ten, eleven, or twelve metabolites in Table 1 can be quantified and used for these purposes. In some embodiments, any thirteen or fourteen metabolites in Table 1 can be quantified and used for these purposes. In some embodiments, all the metabolites in Table 1 can be quantified and used for these purposes.

In some embodiments, the one or more target metabolites for detecting resectable PDAC and/or facilitating the treatment of PDAC may include at least one metabolite of Table 1 and at least one metabolite of Table 2. Each of the metabolites in Table 2 is found to be closely correlated with the presence of resectable PDAC. In some embodiments, the one or more target metabolites may further include 1, 2, 3, or 4 metabolites of the metabolites in Table 2. For example, the one or more target metabolites may include one metabolite in Table 1 and one metabolite in Table 2. As another example, the one or more target metabolites may include one metabolite in Table A and two metabolites in Table B. As yet another example, the one or more target metabolites may include two metabolites in Table 1 and one metabolite in Table 2. Similarly, any combinations of one or more metabolites in Table 1 and one or more metabolites in Table 2 may be used to achieve the same purposes. In some embodiments, one or more of the metabolites in Table 2 may be used, independently from the metabolites listed in Table 1, for detecting and/or facilitating the treatment of resectable PDAC in the subject.

TABLE 2

| | | MASS | Delta |
|---|---|---|---|
| Meta ID | Compound | (+/−) | (ppm) |
| YX009 | C15H15NO4 | 315.134(+) | 0 |
| YX013 | C26H43NO8S | 571.312(+) | 13 |
| YX074 | C29H56NO9P | 628.325(−) | 22 |

In some embodiments, the one or more target metabolites provided by the present disclosure may include at least one metabolite of Table 3. Each of the metabolites in Table 3 is found to be correlated with the presence of resectable PDAC. In some embodiments, one or more of the metabolites in Table 3 may be used, in addition to the one or more metabolites listed in Table 1 and/or one or more metabolites listed in Table 2, for detecting resectable PDAC and/or facilitating the treatment of resectable PDAC in the subject. In some embodiments, the abundance of 1, 2, 3, 4, 5, 6, 7, 8, 9, or 10 metabolites of the metabolites in Table 3 may be quantified for the same purposes.

TABLE 3

| | | MASS | Delta |
|---|---|---|---|
| Meta ID | Compound | (+/−) | (ppm) |
| YX003 | C6H7N5O | 166.072(+) | 2 |
| YX021 | C21H42NO7P | 452.275(+) | 5 |
| YX047 | C36H44N4O8 | 625.303(+) | 0 |
| YX058 | C30H59O8P | 613.359(−) | 8 |
| YX068 | C31H53O9P | 581.33(−) | 10 |
| YX081 | C22H46NO7P | 512.297(−) | 5 |
| YX087 | C25H42NO7P | 498.263(−) | 1 |
| YX099 | C29H39NO4 | 500.257(−) | 1 |
| YX112 | C25H48NO7P | 504.322(−) | 25 |
| YX128 | C23H46NO7P | 478.292(−) | 4 |

It should be noted that one or more of the metabolites listed in Table 1-3 may have one or more isomeride forms, which are included in the scope of the group of diagnostic biomarkers provided by the present disclosure.

According to another aspect of the present disclosure, a method of detecting resectable PDAC in a subject is provided. In some embodiments, the method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has resectable PDAC by comparing the sample score to a cut-off score. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-3.

In some embodiments, the abundance of the one or more components of the panel of the plurality of metabolites may be measured using mass spectrometry (MS; e.g., liquid chromatography-mass spectrometry (LC-MS), gas chromatography-mass spectrometry (GC-MS); matrix-assisted laser desorption/ionization time-of-flight mass spectrometry (MALDI-TOF MS)), ultraviolet spectrometry, High-Performance Liquid Chromatography (HPLC), or the like. In some embodiments, step b) may further include normalizing the abundance of each of the metabolites quantified in step (a), and determining the sample score by processing the normalized abundance with a prediction model.

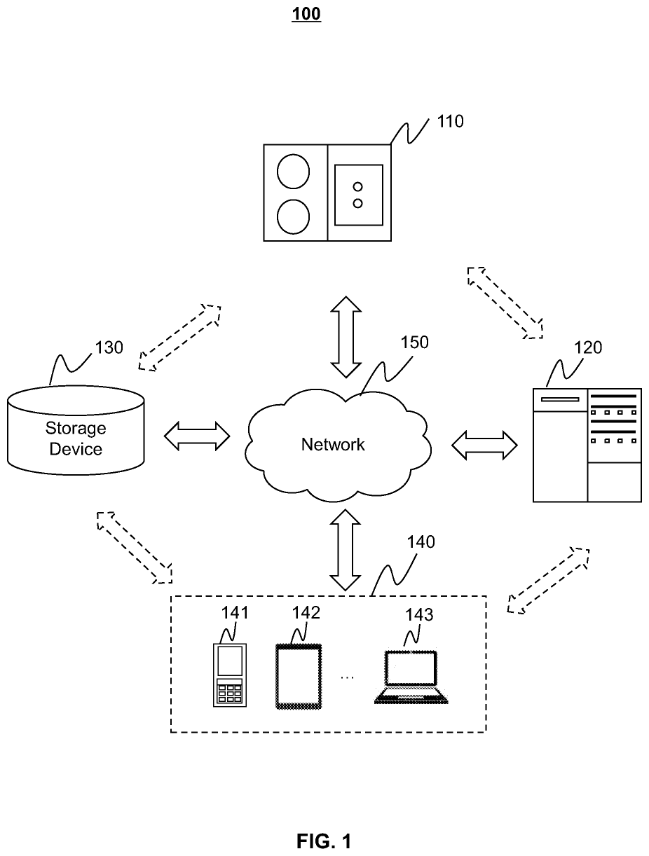

In some embodiments, the determination of the sample score may be implemented on a computing device (e.g., the processing device 120 illustrated in FIG. 1). The computing device may obtain a prediction model for determining the sample score. The abundance of each of the metabolites quantified in step a) may be inputted into the prediction model. The prediction model may process the abundance (e.g., a relative abundance or an absolute abundance) of each of the metabolites quantified in step a) and output the sample score. Merely by way of example, the abundance of each of the metabolites quantified in step a) may be quantified by measuring the concentration of each of the metabolites. In some embodiments, the measured concentration may be normalized. For instance, the measured concentration may be divided by a total concentration of all metabolites in the sample. The sample score may indicate a probability that the subject has PDAC.

In some embodiments, the prediction model may be a trained machine-learning model. For example, the prediction model may be generated using a gradient boosting decision tree (GBDT) algorithm, a decision tree algorithm, a Random Forest algorithm, a logistic regression algorithm, a support vector machine (SVM) algorithm, a Naive Bayesian algorithm, an AdaBoost algorithm, a K-a nearest neighbor (KNN) algorithm, a Markov Chains algorithm, an XGBoosting algorithm, a deep learning algorithm, a neural network, or the like, or any combination thereof, which is not limited by the present disclosure.

To obtain the prediction model, a preliminary model may be trained using a plurality of training datasets. Each of the plurality of training datasets may include a quantified abundance of a sample metabolite of a reference subject and a label indicating whether the reference subject has resectable PDAC or is normal. The plurality of reference subjects may include a plurality of normal subjects who do not have PDAC, a plurality of subjects having pancreatitis. Merely by way of example, the label may be a positive label or a negative label. The positive label indicates that the reference subject has resectable PDAC, and the negative label indicates that the reference subject is normal, or has a pancreatitis. If a reference subject is not suffering from resectable PDAC, the corresponding label may be designated as 0 (i.e., as a negative label). If a reference subject has PDAC, the corresponding label may be designated as 1 (i.e., as a positive sample). Accordingly, the sample score outputted by the prediction model may be a value between 0 and 1. The closer the sample score is to 1, the higher the probability that the subject has PDAC is.

In step c), the sample score is compared to a cut-off score related to the prediction model. As used herein, the term “cut-off value” refers to a dividing point on measuring scales where evaluation results are divided into different categories. In some embodiments, when the sample score is equal to or greater than the cut-off score, the computing device may determine that the subject has PDAC. The cut-off value may be determined based on the performance of the prediction model. In some embodiments, the cut-off value may be a value between 0.37-0.52. In some embodiments, the cut-off value may be a value between 0.40-0.46. In some embodiments, the cut-off value may be a value between 0.45-0.55. For example, the cut-off value may be 0.37, 0.40, 0.41, 0.42, 0.43, 0.45, 0.46, 0.52, 0.55, etc.

In some embodiments, the prediction model may be used to distinguish normal people from resectable PDAC patients in different stages. In some embodiments, the plurality of reference subjects having resectable PDAC may include a plurality of subjects having stage I of PDAC. In some embodiments, the plurality of reference subjects having PDAC may include a plurality of subjects having stage II of PDAC. In some embodiments, the prediction model for detecting stage I and stage II PDAC may be established in a manner similar to the prediction model for detecting resectable PDAC as described earlier in the present disclosure.

In some embodiments, a receiver operating characteristic (ROC) curve may be used to evaluate the performance of the prediction model. The ROC curve may illustrate the diagnostic ability of the prediction model as its cut-off value is varied. The ROC curve is usually generated by plotting the sensitivity against the specificity. An area-under-the-curve (AUC) may be determined based on the ROC curve. The AUC may indicate the probability that a classifier (i.e., the prediction model) will rank a randomly chosen positive instance higher than a randomly chosen negative one.

In some embodiments, the AUC of the prediction model provided by the present disclosure is more than 0.55. In some embodiments, the AUC of the prediction model provided by the present disclosure is more than 0.70. In some embodiments, the AUC of the prediction model provided by the present disclosure is more than 0.75. In some embodiments, the AUC of the prediction model provided by the present disclosure is more than 0.8. In some embodiments, the AUC of the prediction model provided by the present disclosure is more than 0.85. In some embodiments, the AUC of the prediction model provided by the present disclosure is more than 0.9. In some embodiments, the AUC of the prediction model provided by the present disclosure is more than 0.95.

In some embodiments, the sensitivity of the prediction model for detecting resectable PDAC is equal to or greater than 30%. In some embodiments, the sensitivity of the prediction model for detecting resectable PDAC is equal to or greater than 50%. In some embodiments, the sensitivity of the prediction model for detecting resectable PDAC is equal to or greater than 60%. In some embodiments, the sensitivity of the prediction model for detecting resectable PDAC is equal to or greater than 70%. In some embodiments, the sensitivity of the prediction model for detecting resectable PDAC is equal to or greater than 80%. In some embodiments, the sensitivity of the prediction model for detecting resectable PDAC is equal to or greater than 100%.

In some embodiments, the specificity of the prediction model for detecting resectable PDAC is equal to or greater than 60%. In some embodiments, the specificity of the prediction model for detecting resectable PDAC is equal to or greater than 70%. In some embodiments, the specificity of the prediction model for detecting resectable PDAC is equal to or greater than 75%. In some embodiments, the specificity of the prediction model for detecting resectable PDAC is equal to or greater than 80%. In some embodiments, the specificity of the prediction model for detecting resectable PDAC is equal to or greater than 90%. In some embodiments, the specificity of the prediction model for detecting resectable PDAC is equal to or greater than 95%.

More descriptions regarding the performance of some exemplary prediction models for detecting resectable PDAC may be found in the Examples section.

FIG. 1 is a schematic diagram illustrating an exemplary system for detecting PDAC in a subject according to some embodiments of the present disclosure. In some embodiments, the method for detecting PDAC in a subject may be implemented on the system 100. As illustrated, the system 100 may include a quantitative measurement device 110, a processing device 120, a storage device 130, a terminal device 140, and a network 150. The components of the system 100 may be connected in various ways. Merely by way of example, as illustrated in FIG. 1, the quantitative measurement device 110 may be connected to the processing device 120 directly as indicated by the bi-directional arrow in dotted lines linking the quantitative measurement device 110 and the processing device 120, or through the network 150. As another example, the storage device 130 may be connected to the quantitative measurement device 110 directly as indicated by the bi-directional arrow in dotted lines linking the quantitative measurement device 110 and the storage device 130, or through the network 150. As still another example, the terminal device 140 may be connected to the processing device 120 directly as indicated by the bi-directional arrow in dotted lines linking the terminal device 140 and the processing device 120, or through the network 150.

The quantitative measurement device 110 may be configured to measure an abundance of one or more target metabolites for detecting whether the subject has resectable PDAC. In some embodiments, the quantitative measurement device 110 may measure the abundance of the one or more target metabolites using a relative quantification approach or an absolute quantification approach. Merely by way of example, the quantitative measurement device 110 may include a mass spectrometer (MS; e.g., liquid chromatography-mass spectrometer, gas chromatography-mass spectrometer; matrix-assisted laser desorption/ionization time-of-flight mass spectrometer), an ultraviolet spectrometer, a High-Performance Liquid Chromatography (HPLC) apparatus, or the like.

The processing device 120 may process data and/or information obtained from the quantitative measurement device 110, the storage device 130, and/or the terminal device 140. In some embodiments, the processing device 120 may be used to process the quantified abundance of the one or more target metabolites for evaluating whether the subject has PDAC. For example, the processing device 120 may obtain a prediction model. The quantified abundance of the one or more target metabolites may be inputted into the prediction model to obtain a sample score for the subject. The processing device 120 may further evaluate whether the subject has resectable PDAC by comparing the sample score to a cut-off value of the prediction model. In some embodiments, the processing device 120 may determine the quantified abundance of the one or more target metabolites based on data acquired by the quantitative measurement device 110.

In some embodiments, the processing device 120 may be a single server or a server group. The server group may be centralized or distributed. In some embodiments, the processing device 120 may be local or remote. For example, the processing device 120 may access information and/or data from the quantitative measurement device 110, the storage device 130, and/or the terminal device 140 via the network 150. As another example, the processing device 120 may be directly connected to the quantitative measurement device 110, the terminal device 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal device 140. In some embodiments, the processing device 120 may be part of the quantitative measurement device 110.

The storage device 130 may store data, instructions, and/or any other information. In some embodiments, the storage device 130 may store data obtained from the quantitative measurement device 110, the processing device 120, and/or the terminal device 140. The data may include quantified abundance of the one or more target metabolites of the subject and/or the prediction model for processing the quantified abundance, etc. In some embodiments, the storage device 130 may store data and/or instructions that the processing device 120 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. Some embodiments, the storage device 130 may be connected to the network 150 to communicate with one or more other components (e.g., the processing device 120, the terminal device 140) of the system 100. One or more components of the system 100 may access the data or instructions stored in the storage device 130 via the network 150. In some embodiments, the storage device 130 may be integrated into the quantitative measurement device 110 or the processing device 120.

The terminal device 140 may be connected to and/or communicate with the quantitative measurement device 110, the processing device 120, and/or the storage device 130. In some embodiments, the terminal device 140 may include a mobile device 141, a tablet computer 142, a laptop computer 143, or the like, or any combination thereof. For example, the mobile device 141 may include a mobile phone, a personal digital assistant (PDA), or the like, or any combination thereof. In some embodiments, the terminal device 140 may include an input device, an output device, etc. The input device may include alphanumeric and other keys that may be input via a keyboard, a touchscreen (e.g., with haptics or tactile feedback), a speech input, an eye-tracking input, a brain monitoring system, or any other comparable input mechanism. Other types of input devices may include a cursor control device, such as a mouse, a trackball, or cursor direction keys, etc. The output device may include a display, a printer, or the like, or any combination thereof. The terminal device 140 may be used to present information to a user and/or convey a user instruction to other components of the system 100. For example, the user (e.g., a doctor) may instruct the quantitative measurement device 110 to start quantifying the abundance of the one or more metabolites via the terminal device 140. As another example, the user may view an evaluation result regarding whether the subject has resectable PDAC via the terminal device 140.

The network 150 may include any suitable network that can facilitate the exchange of information and/or data for the system 100. In some embodiments, one or more components (e.g., the quantitative measurement device 110, the processing device 120, the storage device 130, the terminal device 140) of the system 100 may communicate information and/or data with one or more other components of the system 100 via the network 150.

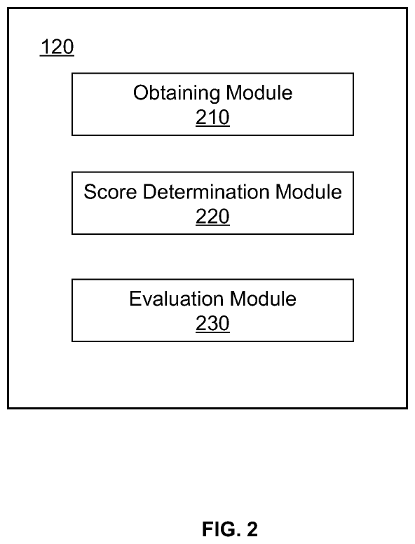

FIG. 2 is a block diagram illustrating an exemplary processing device according to some embodiments of the present disclosure. In some embodiments, the processing device 120 may include an obtaining module 210, a score determination module 220, and an evaluation module 230. In some embodiments, the modules may be hardware circuits of all or part of the processing device 120. The modules may also be implemented as an application or set of instructions read and executed by the processing device 120. Further, the modules may be any combination of the hardware circuits and the application/instructions. For example, the modules may be part of the processing device 120 when the processing device 120 is executing the application/set of instructions. In some embodiments, the processing device 120 may include a processor implemented on the terminal device 140.

The obtaining module 210 may obtain, from a quantitative measurement device (e.g., the quantitative measurement device 110 in FIG. 1), quantified abundance of one or more target metabolites in a panel of a plurality of metabolites in a sample from the subject.

The score determination module 220 may determine a sample score by processing the quantified abundance of each of the one or more target metabolites using a prediction model.

The evaluation module 230 may estimate whether the subject has resectable PDAC by comparing the sample score to a cut-off score.

According to yet another aspect of the present disclosure, a method of detecting PDAC in a subject is provided. The method may include: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; (c) evaluating whether the subject has PDAC by at least comparing the sample score to a cut-off score. In some embodiments, the method may be performed by the processing device 120 and/or one or more modules illustrated in FIG. 2.

In some embodiments, the subject is human. The sample may be a blood serum sample.

In some embodiments, the method of detecting resectable PDAC in a subject may be used for discriminating subjects having resectable PDAC/at a high risk of having PDAC from normal subjects. The subjects having PDAC may be in the stage I of PDAC, or stage II of PDAC.

In some embodiments, the method may further include treating the subject. For example, the method may include steps (a)-(c) and further includes step (d): in response to determining that the subject has resectable PDAC, applying a treatment to the subject. The treatment may include a surgery, radiation therapy, chemotherapy, targeted therapy, or immunotherapy in the subject, or the like or any combination thereof. In some embodiments, surgery can significantly extend survival of PDAC, and surgical options may include a Whipple procedure (pancreaticoduodenectomy) to remove the head of the pancreas along with the surrounding structures, distal pancreatectomy to remove the tail and body of the pancreas, or total pancreatectomy in rare cases where the entire pancreas is removed. Radiation therapy may include high-energy X-rays or other radiation forms, which are used to target and kill cancer cells, and can be used before surgery (neoadjuvant) to shrink tumors or after surgery (adjuvant) to destroy any remaining cancer cells. Anti-cancer medications are given either orally or intravenously to kill cancer cells throughout the body and can be used before or after surgery. Certain drugs are designed to target specific abnormalities or genetic mutations in cancer cells, disrupting their growth and survival. Immunotherapy helps to boost the subject's immune system to recognize and destroy cancer cells. Chemotherapy has long been the backbone of pancreatic cancer management. Exemplary chemotherapy includes gemcitabine, paclitaxel/nab-paclitaxel, 5-fluorouracil, irinotecan, oxaliplatin, etc. The gemcitabine is commonly used chemotherapy drug. The standard dose for gemcitabine may be about, for example, 1000 mg/m², which is administered intravenously over 30 minutes. It is usually given once a week for several weeks, followed by a week of rest, constituting a cycle. The standard dose of paclitaxel and nab-paclitaxel may be about, for example, 125 mg/m², infused intravenously over 3 hours or over 30 minutes, and both drugs are usually given once a week for several weeks, followed by a week of rest, constituting a cycle. Standard dose for irinotecan may be about, for example, 180 mg/m², infused intravenously over 90 minutes. The standard dose for oxaliplatin may be about, for example, 85 mg/m², infused intravenously over 120 minutes. Targeted therapy aims to specifically target and inhibit certain molecules or pathways involved in the growth and survival of PDAC cancer cells, and may include: Erlotinib, Nab-paclitaxel, HER2 inhibitors, etc. In some embodiments, the above treatment can be used in combination. For example, surgery offers a significant survival benefit to eligible patients, particularly when combined with adjuvant chemotherapy.

The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-3.

In some embodiments, the method for detecting resectable PDAC in a subject and treating the subject may further include: in response to an estimation that the subject has resectable PDAC based on a comparison result of comparing the sample score to the cut-off score, verifying that the subject has resectable PDAC with a diagnostic approach, such as a biopsy test, a computerized tomography (CT) scan, a magnetic resonance imaging (MRI) scan, a positron emission tomography (PET) scan, or the like, or any combination thereof.

In some embodiments, the method for detecting resectable PDAC in a subject provided by the present disclosure may be used as a pre-examination for the subject. For example, according to an evaluation result of resectable PDAC detection in the subject using the one or more target metabolites that the subject is evaluated as having resectable PDAC, approaches for further diagnosis of resectable PDAC may be conducted for the subject.

According to yet another aspect of the present disclosure, a method of detecting stage I of PDAC in a subject is provided. The method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has stage I of PDAC by comparing the sample score to a cut-off score. The description of the one or more target metabolites may be found earlier in the present disclosure. For example, the one or more target metabolites may include at least one, two, three, four, five, six, seven, eight, nine, ten, twelve, or all metabolite in the metabolites of Table 1. As another example, the one or more target metabolites may include at least one metabolite in the metabolites of Table 1, at least one metabolite selected from the metabolites of Table 2-3.

In some embodiments, step b) may further include normalizing the abundance of each of the metabolites quantified in step (a), and determining the sample score by processing the normalized abundance with a prediction model. The prediction model may be established using a plurality of training datasets. For example, each of the plurality of training datasets may include the abundance of a metabolite of a reference subject and a label indicating whether the reference subject has a stage I of PDAC or is normal. The plurality of reference subjects may include a plurality of normal subjects who do not have resectable PDAC and a plurality of subjects having a stage I of PDAC.

According to still another aspect of the present disclosure, a method of detecting stage II of PDAC in a subject is provided. The method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has stage II of PDAC by comparing the sample score to a cut-off score. The description of the one or more target metabolites may be found earlier in the present disclosure. For example, the one or more target metabolites may include at least one, two, three, four, five, six, seven, eight, nine, ten, twelve, or all metabolite in the metabolites of Table 1. As another example, the one or more target metabolites may include at least one metabolite in the metabolites of Table 1, at least one metabolite selected from the metabolites of Table 2-3.

In some embodiments, step b) may further include normalizing the abundance of each of the metabolites quantified in step (a), and determining the sample score by processing the normalized abundance with a prediction model. The prediction model may be established using a plurality of training datasets. For example, each of the plurality of training datasets may include the abundance of a metabolite of a reference subject and a label indicating whether the reference subject has a stage II of PDAC or is normal. The plurality of reference subjects may include a plurality of normal subjects who do not have PDAC and a plurality of subjects having stage II of PDAC.

According to yet another aspect of the present disclosure, a use of one or more target diagnostic biomarkers for generating a trained machine-learning model for estimating whether a subject has resectable PDAC is provided. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-3.

According to still another aspect of the present disclosure, a use of the one or more target metabolites for preparing a kit for detecting resectable PDAC in a subject is provided. The description of the one or more target metabolites may be found earlier in the present disclosure.

According to yet another aspect of the present disclosure, a kit for detecting resectable PDAC in a subject is provided. In some embodiments, the kit may include the one or more reagents, which can detect the target metabolites. The description of the one or more target metabolites may be found earlier in the present disclosure. For example, the one or more reagents may be standard substances for respectively detecting the target metabolites. The standard substances may be used for accurate determination of the abundance of the one or more target metabolites in the subject. Specifically, the standard substances may be used for generating one or more standard curves for quantifying the abundance of the one or more target metabolites in the subject. Additionally, the kit may also include other components which are not limited by the present disclosure, such as one or more quality-control agents, one or more pre-treatment agents for pre-treating the sample of the subject (e.g., a blood serum sample), or the like, or any combination thereof. Additionally or alternatively, the kit may include one or more reagents for detecting levels of the one or more target metabolites.

The methods and metabolite biomarkers provided by the present disclosure are further described according to the following examples, which should not be construed as limiting the scope of the present disclosure. More description regarding the performance of some exemplary prediction models based on the one or more target metabolites may also be found in the following examples. As shown in these Examples, the AUC, specificity, and sensitivity of the prediction models are relatively high, indicating that the prediction model utilizing the abundance of these metabolites may effectively distinguish subjects with resectable PDAC from normal subjects.

### EXAMPLES

### Material and Method

### 1. Study Cohorts and Sample Collection

From 2019 to 2022, 177 consecutive serum samples were enrolled. The discovery cohort was composed of 177 individuals, including 6 normal individuals, 117 pancreatitis patients, and 54 resectable PDAC (stage I and II) patients. This cohort was then divided into a training set (Table 4) for model and cut-off establishment and a testing set (Table 5) for model performance evaluation.

Staging of PDAC individuals are based on the tumor size, node, metastasis staging system maintained by the American Joint Committee on Cancer and the International Union for Cancer Control. CA19-9 is a mucin-type glycoprotein tumor marker and is the most sensitive marker reported to date for pancreatic cancer. In Table 4 and Table 5, the upper limit of the normal reference range (37 U/ml) is used as the diagnostic criterion.

TABLE 4
Cohort composition and baseline information of the training set.

| | PDAC | pancreatitis | N |
|---|---|---|---|
| Num | | | |
| Total | 36 | 63 | 6 |
| Male | 16 | 41 | 4 |
| Female | 20 | 22 | 2 |
| Age | | | |
| Average ± SD | 61.5 ± 10.60 | 57.7 ± 9.70 | 59.5 ± 13.52 |
| CA19-9 | | | |
| (cut-off: 37 U/ml) | | | |
| Positive | 26 | 3 | 0 |
| Negative | 7 | 47 | 4 |
| (Unknown) | 3 | 13 | 2 |
| Stage | | | |
| I | 7 | \ | \ |
| II | 29 | \ | \ |

TABLE 5
Cohort composition and baseline information of the testing set.

| | PDAC | pancreatitis | N |
|---|---|---|---|
| Num | | | |
| Total | 18 | 54 | 0 |
| Male | 8 | 37 | 0 |
| Female | 10 | 17 | 0 |
| Age | | | |
| Average ± SD | 59.7 ± 10.69 | 59.2 ± 9.32 | — |
| CA19-9 | | | |
| (cut-off: 37 U/ml) | | | |
| Positive | 13 | 6 | 0 |
| Negative | 5 | 48 | 0 |
| (Unknown) | 0 | 0 | 0 |
| Stage | | | |
| I | 6 | \ | \ |
| II | 12 | \ | \ |

### 2. Reagents and Equipment

### Equipment

Vortex mixer (Kylin-Bell Vortex X5)
20 μL, 100 μL, 200 μL, 1000 μL Pipettes and tips (Gilson)
High-speed microcentrifuge (Centrifuge 5415R)
Electronic balance (MettlerToledo AB104)
Centrifugal vacuum evaporator (TOMY CC-105)
Exion −20adxr Ultra Performance Liquid Chromatography system (shimadzu) coupled with a Triple Quad™ 4500MD LC-MS/MS system (AB Sciex)
ACQUITY UPLC BEH C18 Column (Shim-pack Velox C18 2.7 μm 2.1×100 mm)
R statistical scripting language (version 3.6.1)
AB Sciex Analyst software system (version 1.6.3)

### Reagents and Supplies

LC-MS-grade methanol (Thermo Fisher Scientific)
LC-MS-grade acetonitrile (Thermo Fisher Scientific)
LC-MS-grade formic acid (Thermo Fisher Scientific)
Ammonium acetate, LC-MS grade (Thermo Fisher Scientific)
¹³C cholic acid (Sigma-Aldrich)
Ultrapure water, HPLC grade (watsons)
Centrifuge tubes (1.5 mL; Axygen, cat. no. MCT-150-C)
10 μL, 200 μL, 1000 μL Pipette tips (Axygen)

### Solutions

¹³C labeled cholic acid stock solution (internal standard): weigh 10.8 mg cholic acid and dissolve into 1080 μL methanol, violently vortex until total dissolution. The final concentration of the stock solution is 10 mg/mL.

Precipitation solution: add 120 μL ¹³C cholic acid stock solution into 300 mL methanol and mix.

### 3. Targeted Metabolomics Detection

### I. Metabolites Extraction

For metabolite extraction in targeted metabolomics detection, 10 μL internal standard solution (5 g/mL ¹³C-Cholic Acid) was added to 80 μL serum with 150 μL acetonitrile: isopropanol (4:1 by volume, Thermo Fisher), 50 μL ammonium formate (0.5 g/mL), vortexing and followed by centrifugation at 17,949 g for 5 min. Then, 60 μL supernatant was diluted with 150 μL HPLC-grade water before use.

### II. Detection Method

The pseudo-targeted method in dependent of pure standards was developed, similar to what has been described by Fujian Zheng et. al (Nature Protocols, 2020), determining relative level of all metabolites in the identified panel by using the same reference pool sample for normalizing abundances for each individual. Targeted metabolomics detection was carried on AB SCIEX Triple Quad™ 4500 system and run-in separate ion modes (positive and negative). The mobile phase and the column used for reversed-phase liquid chromatography were used as listed in the table below. The injection volume was 15 μL for each mode. Metabolites were eluted from the column at a flow rate of 0.3 mL/min with a gradually increasing concentration of mobile phase B, 12% of mobile phase B initially, to 60 of the mobile phase B after 2.5 min. A linear 60%-85% and 85%-100% phase B gradient was set at 6 min and 8.5 min Delustering potentials and collision energies were optimized from the quality control samples of the control group. Metabolite peaks were integrated using the Sciex Analyst 1.6.3 software.

### III. Chromatography Parameters

TABLE 6
Details of chromatography parameters.
Positive mode

| Column | Shimadzu shim-pack Velox C18(50*2.1 mm, 2.7 μm) | | | |
|---|---|---|---|---|
| Temperature | 45 °C | | | |
| Autosampler | 15 °C | | | |
| temperature | | | | |
| Mobile Phase | A: 0.1% formic acid-water | | | |
| | B: 0.1% formic acid-acetonitrile | | | |
| | | flow | mobile | mobile |
| | | rate(ml/ | phase | phase |
| | time(min) | min) | A (%) | B (%) |
| Mobile phase | 0.50 | 0.2-0.5 | 65-85 | 35-15 |
| gradient | 2.50 | 0.2-0.5 | 40-70 | 60-30 |
| | 3.70 | 0.2-0.5 | 15-25 | 85-75 |
| | 3.71 | 0.2-0.5 | 1-10 | 99-90 |
| | 4.99 | 0.2-0.5 | 1-10 | 99-90 |
| | 5.00 | 0.2-0.5 | 70-90 | 20-10 |
| | 6.00 | 0.2-0.5 | 70-90 | 30-10 |

| | |
|---|---|
| Autosampler | 80% formic acid |
| wash | |
| Sample size | 15 μL |
| Pre-balance | inject wash solution once |
| before sample | |
| injection | |

Negative mode

| Column | Shimadzu shim-pack Velox C18(50*2.1 mm, 2.7 μm) | | | |
|---|---|---|---|---|
| Temperature | 45 °C | | | |
| Autosampler | 15 °C | | | |
| temperature | | | | |
| Mobile phase | A: 0.1% formic acid-water | | | |
| | B: 0.1% formic acid-acetonitrile | | | |
| | | flow | mobile | mobile |
| | | rate(mL/ | phase | phase |
| | time(min) | min) | A (%) | B (%) |
| Mobile phase | 0.10 | 0.2-0.5 | 55-80 | 45-20 |
| gradient | 1.50 | 0.2-0.5 | 55-70 | 45-30 |
| | 2.80 | 0.2-0.5 | 55-70 | 45-30 |
| | 2.81 | 0.2-0.5 | 40-60 | 60-40 |
| | 4.00 | 0.2-0.5 | 40-60 | 60-40 |
| | 6.00 | 0.2-0.5 | 40-60 | 60-40 |
| | 7.00 | 0.2-0.5 | 15-25 | 85-75 |

| | |
|---|---|
| Autosampler | 80% formic acid |
| wash | |
| Sample size | 15 μL |
| Pre-balance | inject wash solution once |
| before sample | |
| injection | |

### IV. Parameters for Mass Spectrum

TABLE 7
details of ion source parameters under positive and negative modes.

| | | | | | |
|---|---|---|---|---|---|
| Curtain Gas | Collision | Ion Spray | Temperature | Ion Source | Ion Source |
| (CUR) | Gas (CAD) | Voltage (IS) | (TEM) | Gas1(Gas1) | Gas2(Gas2) |

Turbo Ion spray (ESI+)

| | | | | | |
|---|---|---|---|---|---|
| 20-40 | 5-10 | 4800-5500 | 400-600 | 40-60 | 40-60 |

Turbo Ion spray (ESI−)

| | | | | | |
|---|---|---|---|---|---|
| 20-40 | 5-10 | −3500-−4500 | 400-600 | 40-60 | 40-60 |

TABLE 8
Scheduled MRM under positive and negative modes.
Turbo Ion spray (ESI+)

| | | | |
|---|---|---|---|
| MRM detection | 40-90 sec | Target Scan Time | 0.2-0.5 sec |

Turbo Ion spray (ESI−)

| | | | |
|---|---|---|---|
| MRM detection | 45-90 sec | Target Scan Time | 0.2-0.5 sec |

### V. Quality Control

### QC Sample

Equal volume (15 uL) of serum derived from each individual from this study was pooled together, and the pooled sample was used as the QC sample. At least 6 QC samples were arranged in each detection batch. Peak areas of metabolites for all individuals were normalized to the same QC sample before subsequent analysis.

### 4. Data Analysis

Data preprocessing, statistical analysis, and predictive model building were conducted using R programming (v3.6.1). Relative abundances for each metabolites were used in this study. Raw abundances of metabolites for all individuals were normalized by Loess, and their ratios to the abundances of the same QC sample were calculated (the relative abundance) and used for subsequent analysis.

### 5. Selection of the Metabolites for PDAC Diagnostic Model

To select the metabolite features for the diagnosis model, the LASSO algorithm with 10-fold cross validation was implemented for feature selection from the serum metabolomics data. The selected feature was subsequently used to construct prediction model by logic regression in the training set, and the cut off value was set at the point to achieve the highest accuracy.

### Example 1 Targeted Metabolomics Detection of Candidate Serum Diagnostic Biomarkers for Resectable PDAC and Feature Selection for Predicting Models

### I. Targeted Metabolomics Detection of Candidate Metabolites

Based on previous studies, 29 metabolites detected via untargeted metabolomics of serum samples were identified, that showed both significant alternations between resectable PDAC and non-PDAC patients. This serum metabolites panel showed potential for discriminating resectable PDAC from normal and pancreatitis individuals. Among them, the MRM detection with the 4500MD UPLC-MS system was utilized to obtain leaps for 29 metabolites, and integrated them into a targeted detection panel (Table 9).

TABLE 9
List of metabolites involved in the PDAC
diagnostic and non-P subtyping panel.

| | | MASS | | |
|---|---|---|---|---|
| NO | Meta ID | (+/−) | Compound | Delta(ppm) |
| 1 | YX003 | 166.072(+) | C6H7N5O | 2 |
| 2 | YX005 | 333.145(+) | C17H21N2O5 | 2 |
| 3 | YX006 | 548.338(+) | C27H46O10 | 9 |
| 4 | YX009 | 315.134(+) | C15H15NO4 | 0 |
| 5 | YX013 | 571.312(+) | C26H43NO8S | 13 |
| 6 | YX017 | 372.321(+) | C21H41NO4 | 27 |
| 7 | YX020 | 302.305(+) | C18H39NO2 | 1 |
| 8 | YX021 | 452.275(+) | C21H42NO7P | 5 |
| 9 | YX039 | 522.362(+) | C26H52NO7P | 13 |
| 10 | YX040 | 522.356(+) | C26H52NO7P | 1 |
| 11 | YX042 | 438.3(+) | C21H44NO6P | 4 |
| 12 | YX047 | 625.303(+) | C36H44N4O8 | 0 |
| 13 | YX050 | 362.113(−) | C12H26NO7P | 3 |
| 14 | YX053 | 331.191(−) | C20H28O4 | 1 |
| 15 | YX054 | 512.299(−) | C22H46NO7P | 1 |
| 16 | YX058 | 613.359(−) | C30H59O8P | 8 |
| 17 | YX068 | 581.33(−) | C31H53O9P | 10 |
| 18 | YX073 | 540.273(−) | C25H39NO6S | 17 |
| 19 | YX074 | 628.325(−) | C29H56NO9P | 22 |
| 20 | YX077 | 536.315(−) | C29H47NO8 | 16 |
| 21 | YX081 | 512.297(−) | C22H46NO7P | 5 |
| 22 | YX087 | 498.263(−) | C25H42NO7P | 1 |
| 23 | YX099 | 500.257(−) | C29H39NO4 | 1 |
| 24 | YX112 | 504.322(−) | C25H48NO7P | 25 |
| 25 | YX126 | 319.228(−) | C20H32O3 | 0 |
| 26 | YX128 | 478.292(−) | C23H46NO7P | 4 |
| 27 | YX131 | 319.229(−) | C20H32O3 | 0 |
| 28 | YX137 | 592.366(−) | C28H54NO7P | 6 |
| 29 | YX147 | 712.3761(−) | C34H64NO10P | 28 |

Note:
MASS refers to mass-to-charge ratio; (−) represents negative charge; (+) represents positive charge.

To further selected serum metabolites that exhibit faithful differences between resectable PDAC and non-PDAC individuals, and establish a reliable non-invasive PDAC detection test by serum metabolites, targeted metabolomics detection of the above 29 metabolites panel were conducted within the training set individuals as described in Table 4.

Among these metabolites, 18 metabolites also showed significant differences between the two groups, and could be precisely detected, with a CV %<15%. As is shown in Table 10 below, CV % values, fold changes and ANOVA p values between resectable PDAC vs. non-PDAC groups for each significantly different metabolite within the targeted metabolite panel were listed.

TABLE 10
CV % values, fold changes and ANOVA p values between resectable
PDAC vs. non-PDAC groups for each metabolite.

| | | ANOVA | | | | ANOVA | |
|---|---|---|---|---|---|---|---|
| | Fold change | p-value | | | Fold change | p-value | |
| | (PDAC vs. | (PDAC vs. | CV (%) of | | (PDAC vs. | (Positive | CV (%) of |
| | non-PDAC | non-PDAC | all QC | | non-PDAC | vs. | all QC |
| Meta ID | groups) | groups) | samples | Meta ID | groups) | negative) | samples |
| YX005 | 2.377 | 0.00010676 | 11.11 | YX050 | 0.007 | 0.0057126 | 5.79 |
| YX006 | 1.115 | 0.034201 | 10.6 | YX053 | 1.221 | 0.02968 | 10.75 |
| YX009 | 1.551 | 0.0012619 | 10.59 | YX054 | 1.627 | 0.00033686 | 12.72 |
| YX013 | 3.791 | 0.035082 | 7.63 | YX073 | 2.042 | 7.0725E−07 | 8.76 |
| YX017 | 1.309 | 0.046544 | 12.59 | YX074 | 2.15 | 2.1649E−06 | 4.1 |
| YX020 | 1.606 | 2.5437E−09 | 11.73 | YX077 | 1.335 | 0.001115 | 4.83 |
| YX039 | 1.278 | 0.00014872 | 10.28 | YX126 | 3.226 | 0.00037046 | 9.21 |
| YX040 | 1.246 | 0.000004746 | 14.75 | YX131 | 1.518 | 0.0096568 | 4.18 |
| YX042 | 1.532 | 1.7672E−06 | 12.67 | YX147 | 0.907 | 0.017032 | 14.56 |

For the metabolite annotations of Meta ID listed in the table above, please refer to “Table 9”.

Metabolites listed in Table 10 were selected and used for subsequent feature selection and model establishment.

### II. Selection of Metabolites Involved in the Prediction Model

Based on these filtered serum metabolites described in above 2 tables, feature selection was performed on the serum metabolomics data of the training cohort using the LASSO algorithm and 10-fold cross-validation to find key metabolite biomarkers for the detection of resectable PDAC (stages I and II). 15 metabolite features have been selected and used for subsequent model construction.

TABLE 11
CV % values, fold changes, ANOVA p values and average
of raw/normalized abundances between resectable
PDAC vs. non-PDAC groups for each metabolite.

| | | | | average | average |
|---|---|---|---|---|---|
| | | | | abundance | abundance |
| | | | | ratio in | ratio in |
| | fold | ANOVA | | PDAC | non-PDAC |
| Meta ID | change | p-value | CV (%) | samples | samples |
| YX005 | 2.377 | 0.00010676 | 11.11 | 2.9905 | 1.2579 |
| YX006 | 1.115 | 0.034201 | 10.6 | 1.1990 | 1.0752 |
| YX017 | 1.309 | 0.046544 | 12.59 | 1.5415 | 1.1773 |
| YX020 | 1.606 | 2.5437E−09 | 11.73 | 1.2738 | 0.7929 |
| YX039 | 1.278 | 0.00014872 | 10.28 | 1.2326 | 0.9647 |
| YX040 | 1.246 | 0.000004746 | 14.75 | 1.2039 | 0.9664 |
| YX042 | 1.532 | 1.7672E−06 | 12.67 | 1.5271 | 0.9971 |
| YX050 | 0.007 | 0.0057126 | 5.79 | 0.0069 | 0.9392 |
| YX053 | 1.221 | 0.02968 | 10.75 | 1.2087 | 0.9900 |
| YX054 | 1.627 | 0.00033686 | 12.72 | 1.5739 | 0.9672 |
| YX073 | 2.042 | 7.0725E−07 | 8.76 | 2.1389 | 1.0476 |
| YX077 | 1.335 | 0.001115 | 4.83 | 1.2123 | 0.9084 |
| YX126 | 3.226 | 0.00037046 | 9.21 | 3.0310 | 0.9394 |
| YX131 | 1.518 | 0.0096568 | 4.18 | 1.5236 | 1.0037 |
| YX147 | 0.87 | 0.018881 | 8.78 | 0.8387 | 0.9639 |

For the metabolite annotations of Meta ID listed in the table above, please refer to “Table 9”.

### Example 2 Performances of the Metabolic Model for Resectable PDAC Patients

### I. Performance of the Metabolic Model in the Training Set

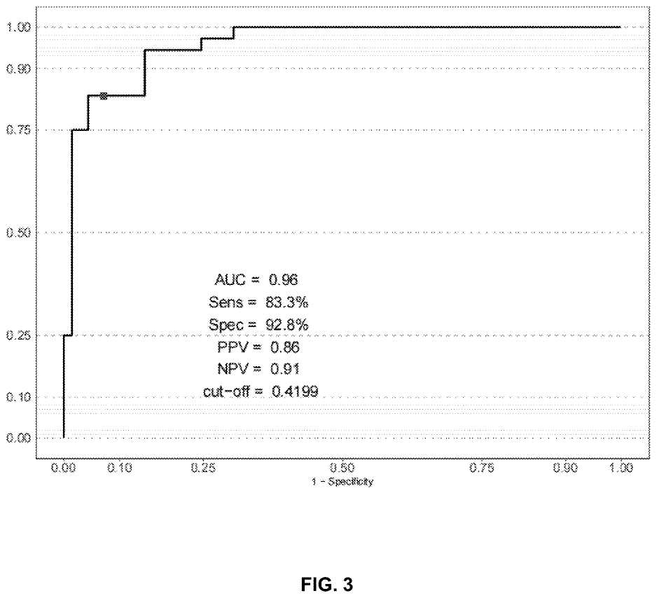

Based on the 15 metabolites selected above, prediction models based on logistic regression in the training set were constructed. As is shown in the FIG. 3, the AUC of this model to discriminate resectable PDAC (in resectable stages:stage I to II) from normal and pancreatitis individuals could achieve 0.96 (sensitivity=83.3%, specificity=92.8%) in the training set at the selected threshold resulting in the highest accuracy (cut-off value=0.4199).

### II. Performance for the Metabolic Model in the Testing Cohort

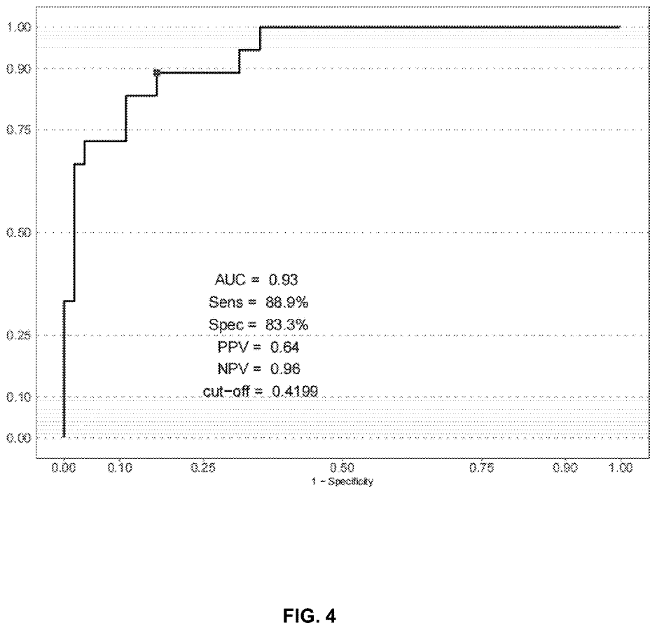

Based on the prediction model constructed in the training cohort and the cut-off value (0.4199), performances of this model in the testing set were further evaluated. The performance of the prediction model for PDAC group (in resectable stages:stage I to II) vs. non-PDAC group were shown in FIG. 4: the AUC was 0.93, with a sensitivity of 88.9%, and a specificity of 83.3%.

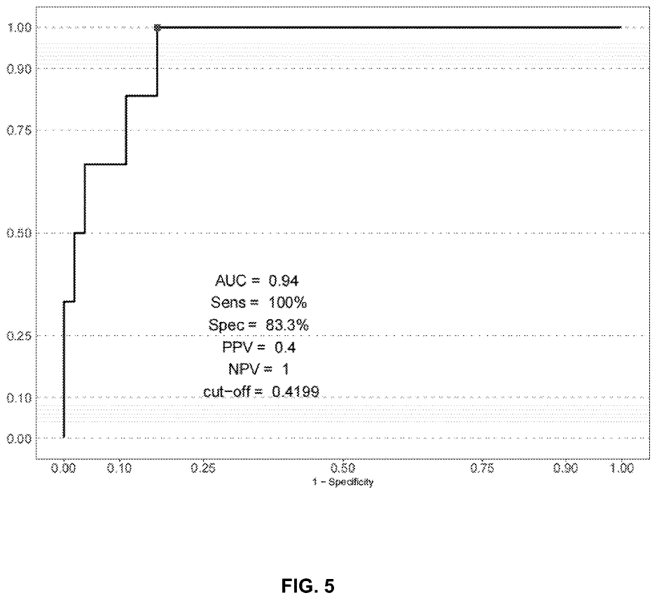

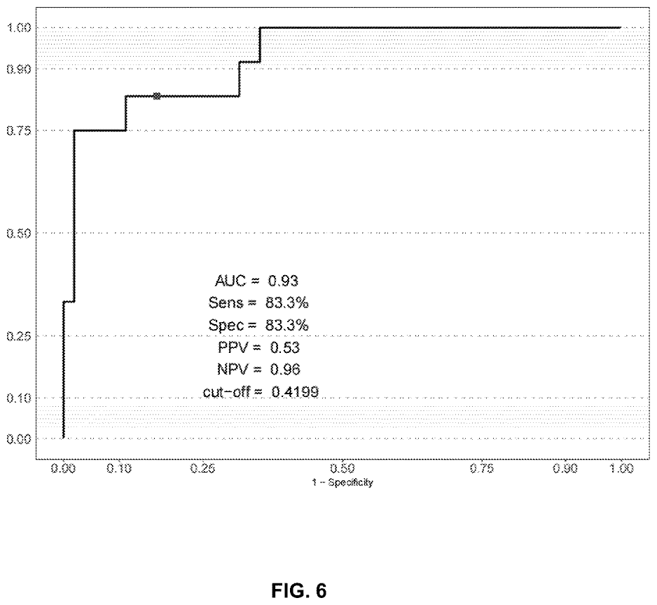

The performance of this model at different stages was evaluated, and found that this model exhibit promising efficiency in discriminating resectable stages (stage I) and resectable stage PDAC (stage II). Specifically, the AUC is 0.94 for resectable PDAC stage I (sensitivity=100%, specificity=83.3%) (see FIG. 5) and 0.93 for resectable PDAC stage II (sensitivity=83.3%, specificity=83.3%) in the testing set (see FIG. 6).

Collectively, the serum metabolites-based model provides a promising non-invasive approach for detection of PDAC, which would greatly favor the treatment and decrease mortality caused by this disease.

III. The Performance of Prediction Models with a Variety of Combinations of the 15 Selected Metabolites

Prediction models based on individual metabolite, or a combination of any two or three within the 15 metabolites panel were also established. Performances of these models were listed in the table below and have been demonstrated in the following sections:

TABLE 12
Performances of prediction models based on individual
metabolites among the selected 15 metabolites.

| Meta ID | AUC | Sensitivity | Specificity | Cut-off |
|---|---|---|---|---|
| YX005 | 0.758 | 0.611 | 0.704 | 0.4052 |
| YX042 | 0.723 | 0.5 | 0.852 | 0.4311 |
| YX073 | 0.757 | 0.556 | 0.833 | 0.4394 |
| YX126 | 0.733 | 0.556 | 0.815 | 0.4009 |

For the metabolite annotations of Meta ID listed in the table above, please refer to “Table 9”.

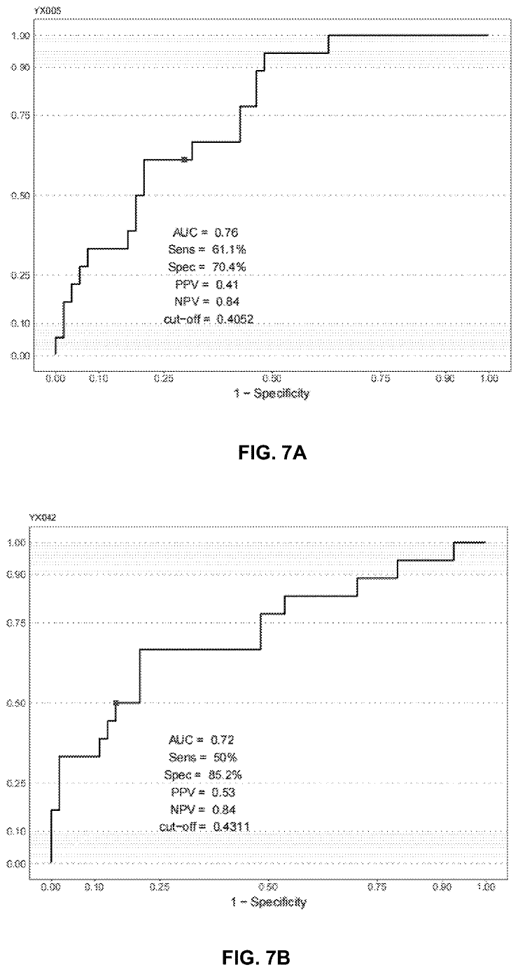

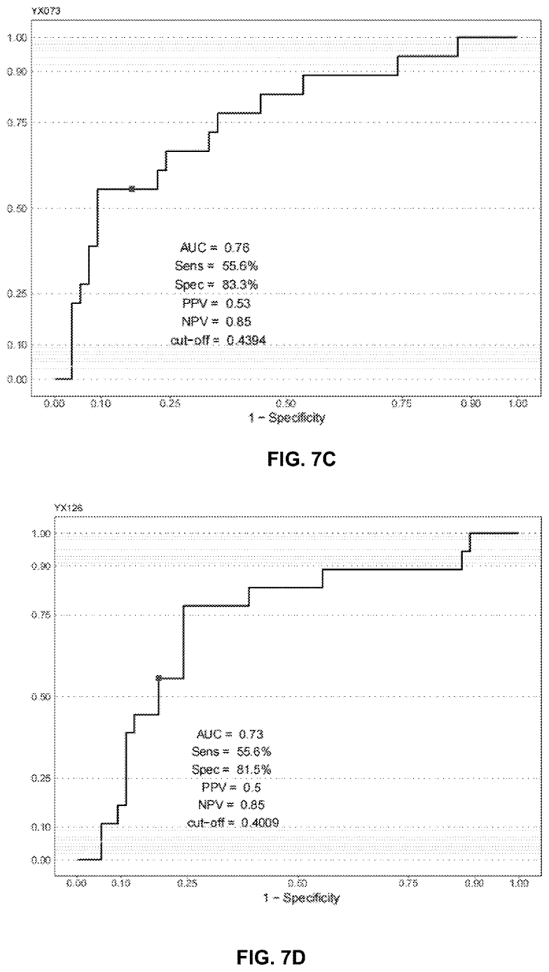

Please see FIGS. 7A-7D for the ROC curve.

TABLE 13
Performances of prediction models based on the combination
of two metabolites among the selected 15 metabolites.

| The combination | | | | |
|---|---|---|---|---|
| of Meta ID1 and | | | | |
| Meta ID2 | AUC | Sensitivity | Specificity | Cut-off |
| YX005, YX042 | 0.834 | 0.778 | 0.759 | 0.4139 |
| YX005, YX050 | 0.82 | 0.667 | 0.722 | 0.4365 |
| YX005, YX073 | 0.813 | 0.778 | 0.704 | 0.3932 |
| YX020, YX073 | 0.817 | 0.667 | 0.852 | 0.4099 |
| YX020, YX131 | 0.801 | 0.333 | 0.926 | 0.5151 |
| YX040, YX126 | 0.801 | 0.722 | 0.833 | 0.3994 |
| YX042, YX126 | 0.805 | 0.556 | 0.889 | 0.4632 |
| YX050, YX073 | 0.808 | 0.611 | 0.833 | 0.4553 |
| YX050, YX126 | 0.817 | 0.722 | 0.87 | 0.3733 |
| YX050, YX131 | 0.803 | 0.611 | 0.833 | 0.4207 |

For the metabolite annotations of Meta ID listed in the table above, please refer to “Table 9”.

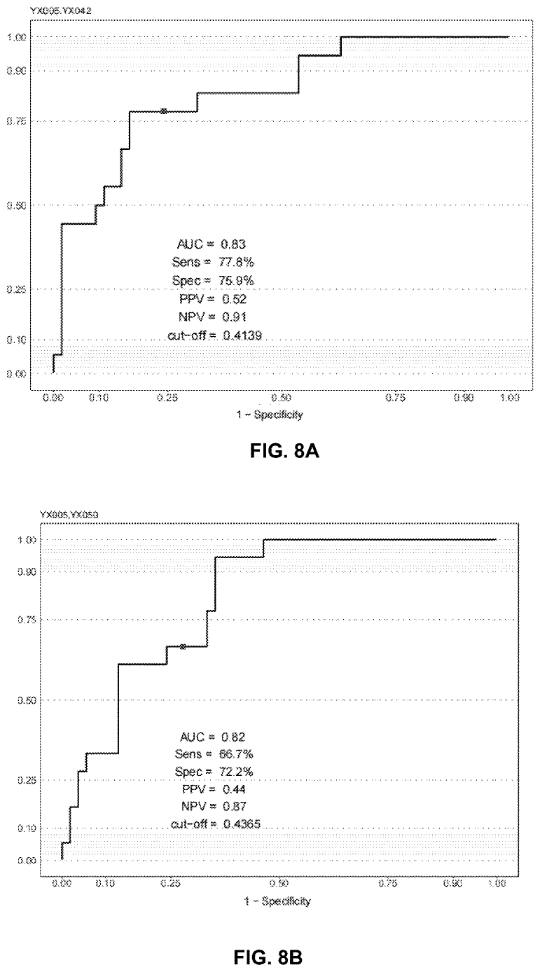

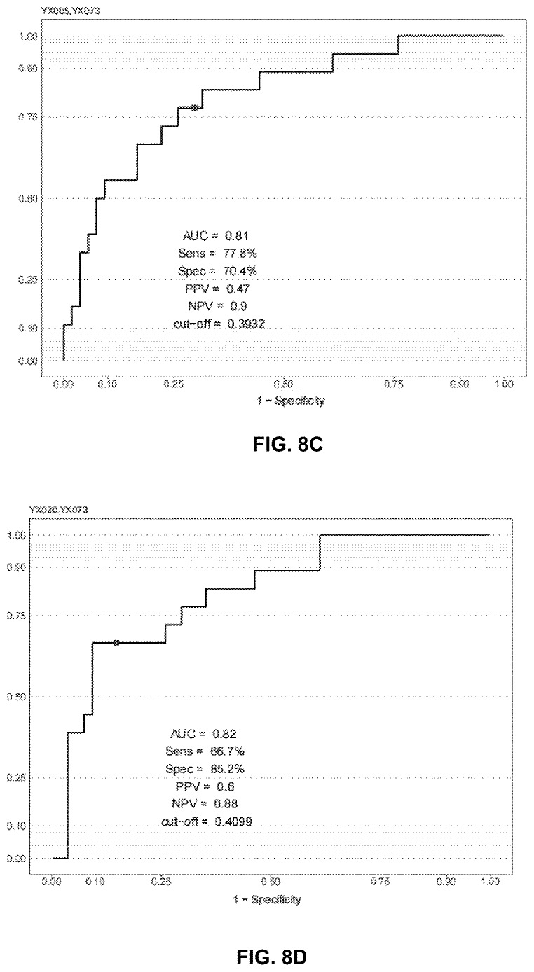

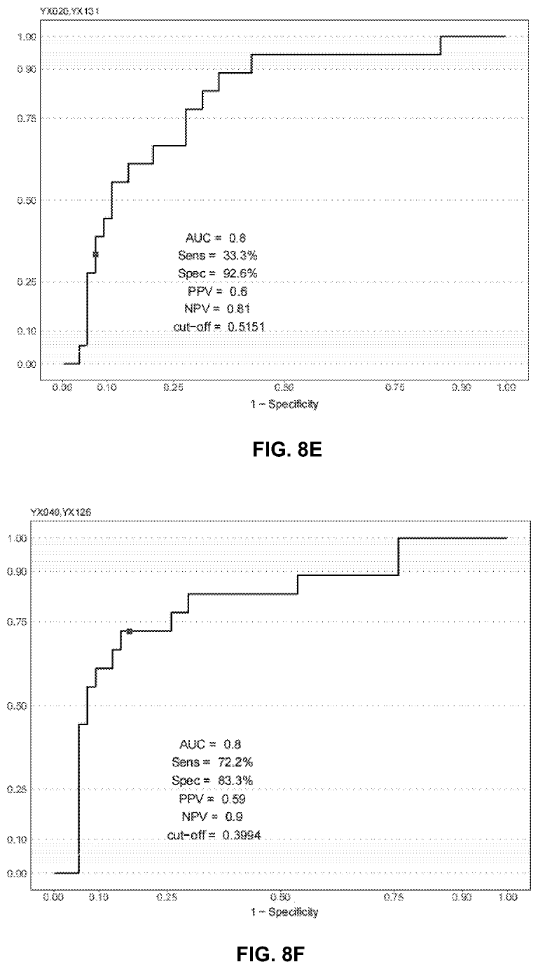

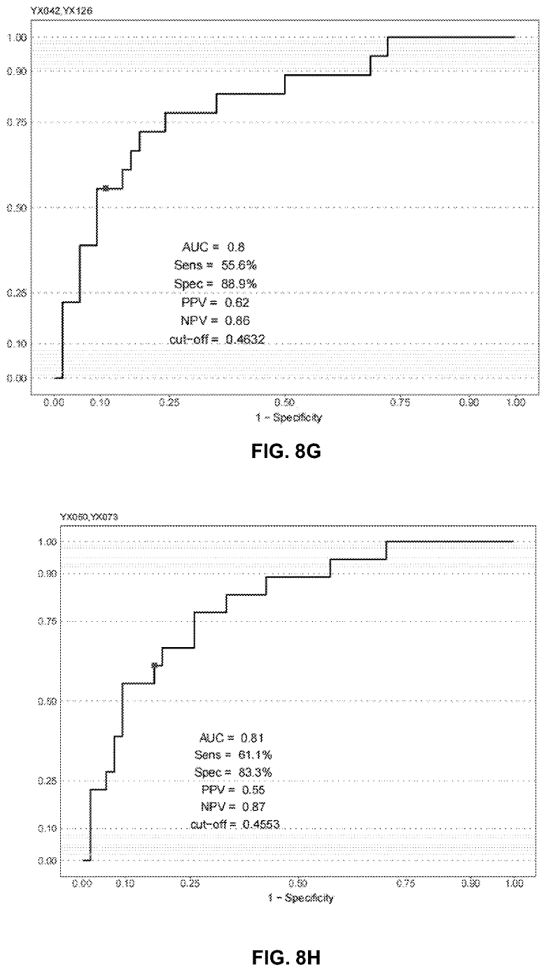

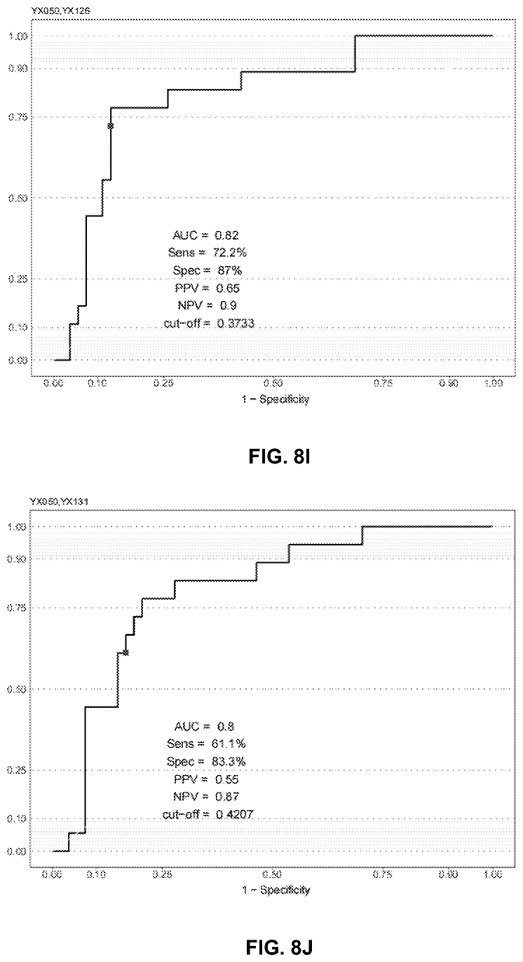

Please see FIGS. 8A-8J for the ROC curve.

TABLE 14
Performances of prediction models based on the combination
of 3 metabolites among the selected 15 metabolites.

| The combination | | | | |
|---|---|---|---|---|
| of Meta ID1 and | | | | |
| Meta ID2 | | | | |
| and Meta ID3 | AUC | Sensitivity | Specificity | Cut-off |
| YX005, YX006, YX042 | 0.848 | 0.722 | 0.796 | 0.459111485 |
| YX005, YX017, YX042 | 0.846 | 0.778 | 0.741 | 0.408599474 |
| YX005, YX039, YX042 | 0.837 | 0.778 | 0.759 | 0.419077002 |
| YX050, YX053, YX147 | 0.831 | 0.5 | 0.889 | 0.555155699 |
| YX050, YX054, YX073 | 0.86 | 0.778 | 0.833 | 0.39954118 |
| YX005, YX042, YX077 | 0.846 | 0.833 | 0.722 | 0.429982325 |

For the metabolite annotations of Meta ID listed in the table above, please refer to “Table 9”

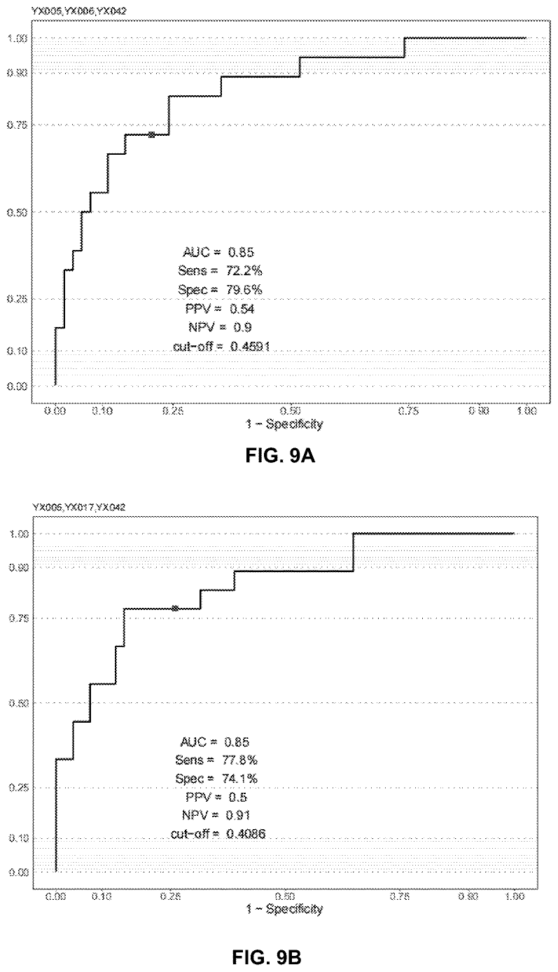

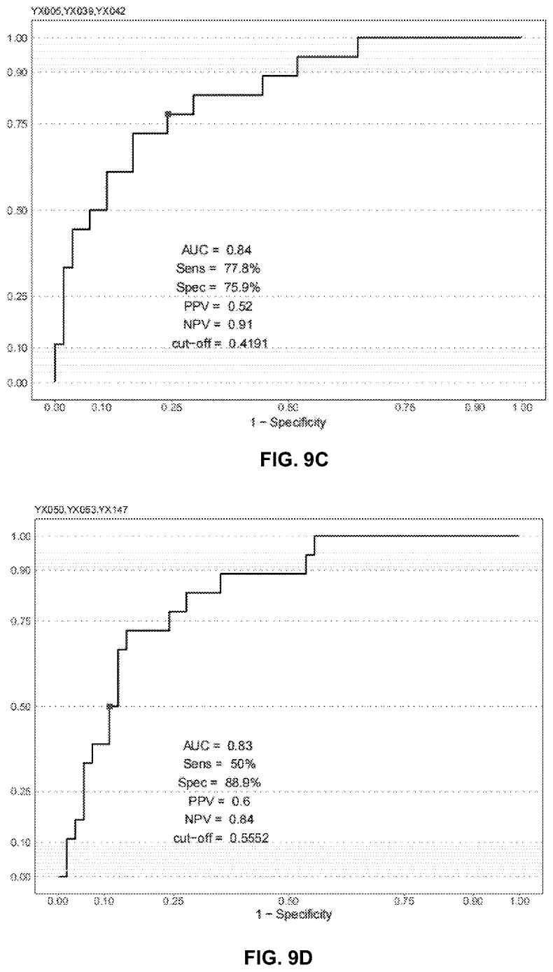

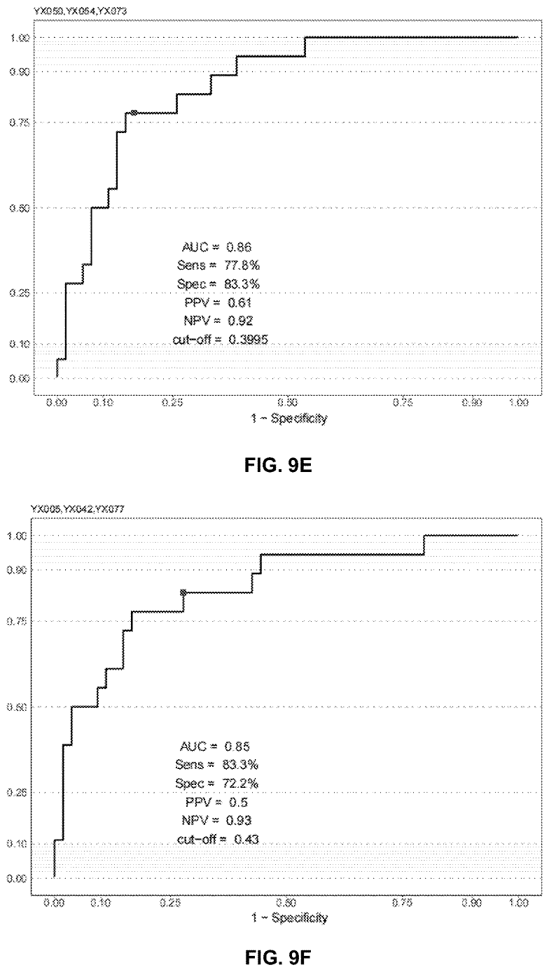

Please see FIGS. 9A-9F for the ROC curve.

### IV. The Performance of CA19-9

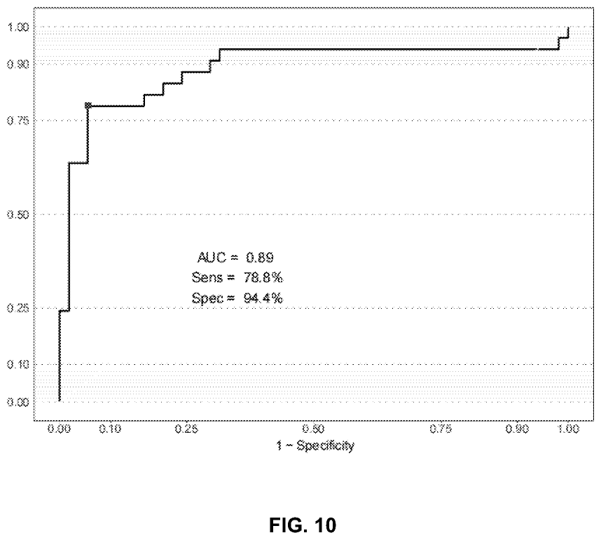

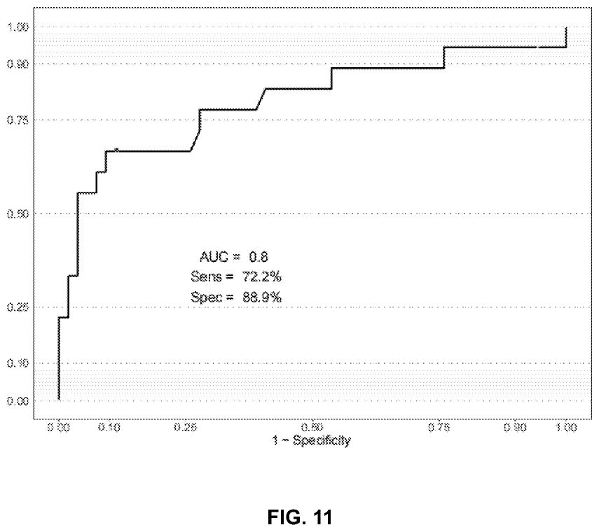

CA19-9 has been recognized as the primary non-invasive biomarker for PDAC diagnosis, but could not be applied to CA19-9 negative population, and the sensitivity and specificity of this biomarker is still limited. Thus, the performance of the CA19-9 were further evaluated. The CA19-9 alone reached an AUC of 0.89 (sensitivity=78.8%, specificity=94.4%) in the training set (see FIG. 10), while 0.8 (sensitivity=72.2%, specificity=88.9%) in the testing set (see FIG. 11), under the clinical used cut-off 37.00 U/ml.

Collectively, a panel of serum metabolites biomarkers was identified, and an MRM based targeted detection assay of these metabolites was developed. Based on these serum metabolites, a promising resectable PDAC (Stage I and Stage II) diagnostic panel was identified, which is significantly more accurate than CA19-9 alone.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

What is claimed is:

1. A system for detecting resectable pancreatic duct adenocarcinoma (PDAC) in a subject, comprising:
at least one storage device including a set of instructions; and
at least one processor in communication with the at least one storage device, wherein when executing the set of instructions, the at least one processor is directed to perform operations including:
(a) obtaining, from a quantitative measurement device, quantified abundance of one or more target metabolites in a panel of a plurality of metabolites in a sample from the subject, wherein the plurality of metabolites include the metabolites of Table A:
TABLE A

| | | MASS | Delta |
|---|---|---|---|
| Meta ID | Compound | (+/−) | (ppm) |
| YX005 | C17H21N2O5 | 333.145(+) | 2 |
| YX006 | C27H46O10 | 548.338(+) | 9 |
| YX017 | C21H41NO4 | 372.321(+) | 27 |
| YX020 | C18H39NO2 | 302.305(+) | 1 |
| YX039 | C26H52NO7P | 522.362(+) | 13 |
| YX040 | C26H52NO7P | 522.356(+) | 1 |
| YX042 | C21H44NO6P | 438.3(+) | 4 |
| YX050 | C12H26NO7P | 362.113(−) | 3 |
| YX053 | C20H28O4 | 331.191(−) | 1 |
| YX054 | C22H46NO7P | 512.299(−) | 1 |
| YX073 | C25H39NO6S | 540.273(−) | 17 |
| YX077 | C29H47NO8 | 536.315(−) | 16 |
| YX126 | C20H32O3 | 319.228(−) | 0 |
| YX131 | C20H32O3 | 319.229(−) | 0 |
| YX147 | C34H64NO10P | 712.3761(−) | 28; |

(b) determining a sample score by processing the quantified abundance of each of the one or more target metabolites using a prediction model; and
(c) estimating whether the subject has resectable PDAC by comparing the sample score to a cut-off score.

2. The system of claim 1, wherein the one or more target metabolites include at least one, two, three, five, eight, twelve metabolites in Table A.

3. The system of claim 1, wherein the one or more target metabolites include all the metabolites in Table A.

4. The system of claim 1, wherein the plurality of metabolites further include metabolites in Table B:
TABLE B

| | | MASS | Delta |
|---|---|---|---|
| Meta ID | Compound | (+/−) | (ppm) |
| YX009 | C15H15NO4 | 315.134(+) | 0 |
| YX013 | C26H43NO8S | 571.312(+) | 13 |
| YX074 | C29H56NO9P | 628.325(−) | 22 |

wherein the one or more target metabolites include at least one metabolite in Table A and at least one metabolite in Table B.

5. The system of claim 1, wherein the plurality of metabolites further include metabolites in Table C:
TABLE C

| | | MASS | Delta |
|---|---|---|---|
| Meta ID | Compound | (+/−) | (ppm) |
| YX003 | C6H7N5O | 166.072(+) | 2 |
| YX021 | C21H42NO7P | 452.275(+) | 5 |
| YX047 | C36H44N4O8 | 625.303(+) | 0 |
| YX058 | C30H59O8P | 613.359(−) | 8 |
| YX068 | C31H53O9P | 581.33(−) | 10 |
| YX081 | C22H46NO7P | 512.297(−) | 5 |
| YX087 | C25H42NO7P | 498.263(−) | 1 |
| YX099 | C29H39NO4 | 500.257(−) | 1 |
| YX112 | C25H48NO7P | 504.322(−) | 25 |
| YX128 | C23H46NO7P | 478.292(−) | 4 |

wherein the one or more target metabolites include at least one metabolite in Table A and at least one metabolite in Table C.

6. The system of claim 1, wherein the quantified abundance of each of the one or more target metabolites is determined by the quantitative measurement device using a relative quantification approach or an absolute quantification approach.

7. The system of claim 1, wherein the sample score indicates a probability that the subject has resectable PDAC.

8. The system of claim 1, wherein the prediction model is a trained machine-learning model, and the trained machine-learning model is obtained by training a preliminary model using a plurality of training datasets, wherein
each of the plurality of training datasets includes quantified abundance of the one or more target metabolites of a reference sample from a reference subject and a label indicating whether or not the reference subject has resectable PDAC.

9. The system of claim 8, wherein the label is a negative label or a positive label, wherein
the positive label indicates that the reference subject has resectable PDAC, and
the negative label indicates that the reference subject is normal or has pancreatitis.

10. The system of claim 1, wherein the PDAC is in stage I or stage II of PDAC.

11. A method of detecting resectable pancreatic duct adenocarcinoma (PDAC) in a subject and treating the subject, comprising:
(a) obtaining, from a quantitative measurement device, quantified abundance of one or more target metabolites in a panel of a plurality of metabolites in a sample from the subject, wherein the plurality of metabolites include the metabolites of Table A;
(b) determining a sample score by processing the quantified abundance of each of the one or more target metabolites using a prediction model;
(c) determining whether the subject has resectable PDAC by at least comparing the sample score to a cut-off score; and
(d) in response to determining that the subject has resectable PDAC, applying a treatment to the subject, wherein the treatment includes a surgery, radiation therapy, chemotherapy, targeted therapy, or immunotherapy.

12. The method of claim 11, wherein the one or more target metabolites include all the metabolites in Table A.

13. The method of claim 11, wherein the plurality of metabolites further include the metabolites in Table B;
wherein the one or more target metabolites include at least one metabolite in Table A and at least one metabolite in Table B.

14. The method of claim 11, wherein the plurality of metabolites further include metabolites in Table C,
wherein the one or more target metabolites include at least one metabolite in Table A and at least one metabolite in Table C.

15. The method of claim 11, wherein the quantified abundance of each of the one or more target metabolites is determined by the quantitative measurement device using a relative quantification approach or an absolute quantification approach.

16. The method of claim 11, wherein the sample score indicates a probability that the subject has resectable PDAC.

17. The method of claim 11, wherein the prediction model is a trained machine-learning model, and the trained machine-learning model is obtained by training a preliminary model using a plurality of training datasets, wherein
each of the plurality of training datasets includes quantified abundance of the one or more target metabolites of a reference sample from a reference subject and a label indicating whether or not the reference subject has resectable PDAC.

18. The method of claim 17, wherein the label is a negative label or a positive label, wherein
the positive label indicates that the reference subject has resectable PDAC, and
the negative label indicates that the reference subject is normal or has pancreatitis.

19. The method of claim 11, wherein the PDAC is in stage I or stage II of PDAC.

20. A kit for detecting resectable pancreatic duct adenocarcinoma (PDAC) in a subject, comprising one or more reagents for quantifying one or more target metabolites in a panel of a plurality of metabolites, wherein the plurality of metabolites include the metabolites in Table A.
